(19) United States
(12) Patent Application Publication
(10) Pub. No.: US 2015/0198587 A1
(43) Pub. Date: Jul. 16, 2015

(54) Detecting Cells in a Cell Suspension

(71) Applicant: SIEMENS AKTIENGESELLSCHAFT, München (DE)

(72) Inventors: Oliver Hayden, Herzogenaurach (DE); Michael Johannes Helou, Regensburg (DE); Lukas Richter, Erlangen (DE)

(21) Appl. No.: 14/409,563

(22) PCT Filed: Jun. 3, 2013
(86) PCT No.: PCT/EP13/61348

§ 371 Date: Dec. 19, 2014

(30) Foreign Application Priority Data
Jun. 22, 2012 (DE) 102012210598.0

(51) Int. Cl. G01N 33/50

(52) U.S. Cl. CPC G01N 33/5094

(57) ABSTRACT

The embodiments relate to an arrangement for quantifying cells. The arrangement includes a magnetic field-sensitive sensor having a first and second pair of sensor elements. The sensor elements of the first pair are connected as part of a Wheatstone bridge and have a first spacing of between half and double a first average size of a first cell or cell conglomerate type. The sensor elements of the second pair are connected as part of a Wheatstone bridge and have a second spacing of between half and double a second average size of a second cell or cell conglomerate type. A third spacing of the two closest sensor elements of the pairs is greater than the larger of the two average sizes. The arrangement also includes a channel for conducting the cell suspension past the sensor elements.

### CROSS-REFERENCE TO RELATED APPLICATIONS

The present patent document is a §371 nationalization of PCT Application Serial Number PCT/EP2013/061348, filed Jun. 3, 2013, designating the United States, which is hereby incorporated by reference, and this patent document also claims the benefit of DE 10 2012 210 598.0, filed on Jun. 22, 2012, which is also hereby incorporated by reference.

### TECHNICAL FIELD

The embodiments relate to a method and to an arrangement for detecting and, more particularly, counting cells in a cell suspension.

### BACKGROUND

The detection of cells and cell interactions within the same blood sample using magnetoresistive methods is an unresolved problem to date. Such interactions, however, are important for medical diagnostics for inferring a particular clinical picture as quickly as possible.

One of these clinical pictures is thrombocytopenia, e.g., the excessively low number of thrombocytes or platelets in blood. Thrombocytopenia may be the result of a blood coagulation disorder or an increased activity of the immune system against the endogenous thrombocytes (e.g., immunothrombocytopenia). A case of immunothrombocytopenia may occur as an autoimmune disease (e.g., immune thrombocytic purpura or idiopathic thrombocytopenic purpura, ITP), in which the inherent immune system detects and removes thrombocytes. Immunothrombocytopenia may also occur when the number of thrombocytes drastically sinks during an infectious disease. In this case, thrombocytes perform tasks within the immune defense process. In this process, the thrombocytes either directly interact with immune cells (e.g., monocytes), forming immune cell/thrombocyte aggregates, or directly interact with the infiltrated microorganisms (e.g., bacteria, viruses, yeasts/fungi). In both cases, the thrombocytes are detected and removed by monocytes. Monocytes are cells of the immune system that circulate in blood, and the precursors of the macrophages localized in, inter alia, the tissues and of a portion of the dendritic cells. Thrombocytes within such aggregates are no longer available for tasks during blood coagulation or hemostasis. The resulting lowering of the thrombocyte count owing to acute immune reactions may be confused with a coagulation disorder. The rapid differentiation of these two clinical pictures (e.g., coagulation disorder or autoimmune disease) may speed up the diagnosis. The present embodiments allow, inter alia, the counting of immune cell/thrombocyte aggregates in whole blood.

To date, the detection of aggregates of immune cells with thrombocytes is, as far as is known, only realized by optical flow cytometry. This technology requires the specific labeling of both cell types (e.g., immune cells and thrombocytes) using antibodies labeled for fluorescence. Furthermore, the optical flow cytometry requires a complex cleanup of the cell types to be investigated or a removal of interfering cell types such as, for example, red blood cells. Without this cleanup, the detection of the fluorescent dyes used would not be possible.

### SUMMARY AND DESCRIPTION

The scope of the present invention is defined solely by the appended claims and is not affected to any degree by the statements within this summary. The present embodiments may obviate one or more of the drawbacks or limitations in the related art.

It is an object of the present embodiments to specify an improved method and a corresponding arrangement for detecting and, more particularly, quantifying cells in a cell suspension, avoiding the disadvantage stated at the beginning.

The arrangement for quantifying cells, while distinguishing between at least two different sizes of cell types and/or cell conglomerate types in a cell suspension, includes a magnetic field-sensitive sensor having at least one first and second pair of sensor elements, wherein (1) the sensor elements of the first pair have a first spacing of between half and double a first mean size of a first type of cell or cell conglomerate to be measured, (2) the sensor elements of the second pair have a second spacing of between half and double a second mean size of a second type of cell or cell conglomerate to be measured, and (3) a third spacing of the sensor elements of the pairs that are closest to one another is greater than the larger of the two mean sizes. The arrangement also includes a channel for guiding the cell suspension past the sensor elements.

It was identified that a specific sensor geometry makes it possible to distinguish between various types of cells and/or conglomerates in a cell suspension. Advantageously, no cleanup or filtering or dilution is required here; instead, the cell suspension may be left in its initial state. Merely a labeling of at least some of the cells with superparamagnetic particles is required in order to generate a signal at the magnetoresistive sensor.

It is useful if the arrangement includes an evaluation unit for evaluating a first signal of the first pair and a second signal of the second pair, the evaluation unit being designed to evaluate both the time lag between the first and the second signal and the amplitude of the two signals.

### BRIEF DESCRIPTION OF THE DRAWINGS

FIG. 1 depicts an example of a measurement system having a fluid channel and giant magnetoresistive (GMR) sensor.

FIG. 2 depicts an example of a conglomerate of monocytes and thrombocytes above the sensor and the associated measured signal.

FIG. 3 depicts an example of a thrombocyte above the sensor and the associated measured signal.

FIG. 4 depicts an example of a medium-sized conglomerate of thrombocytes above the sensor and the associated measured signal.

FIG. 5 depicts an example of a large conglomerate of thrombocytes above the sensor and the associated measured signal.

FIG. 6 depicts an example of a diagram of a GMR sensor in a parallel arrangement in a Wheatstone bridge.

FIG. 7 depicts an example of a diagram of a GMR sensor in a diagonal arrangement in a Wheatstone bridge.

### DETAILED DESCRIPTION

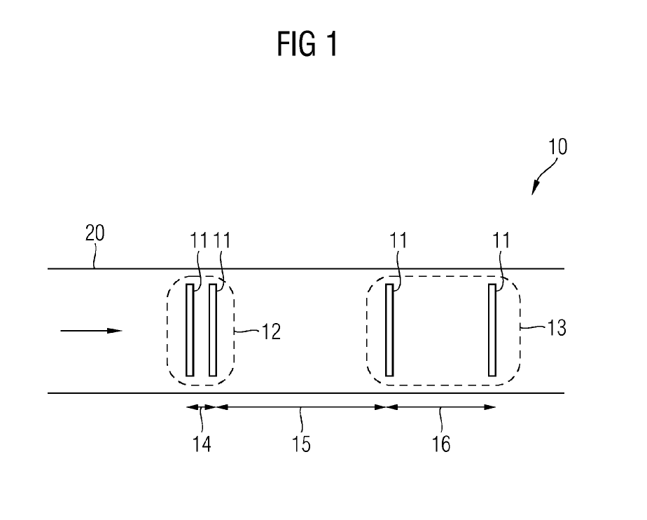

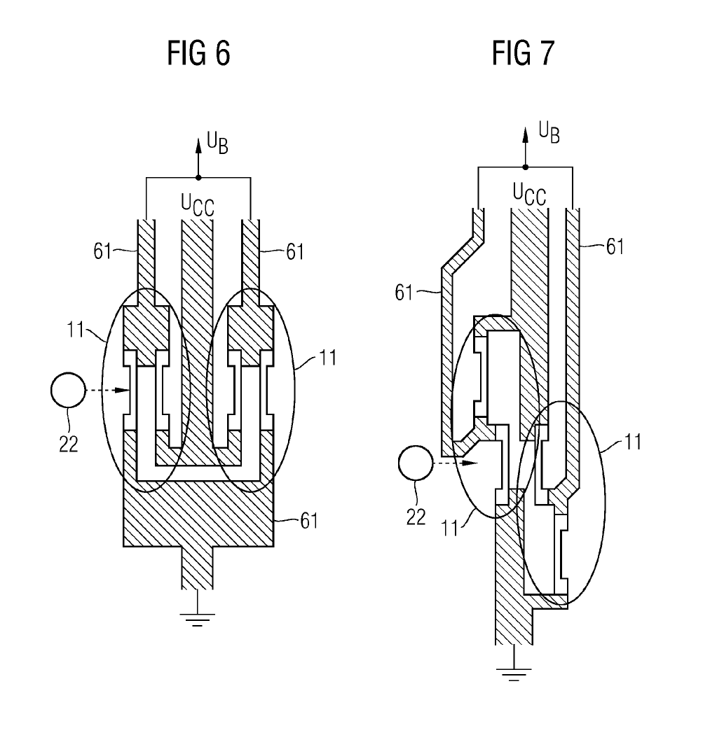

FIG. 1 depicts diagrammatically the fundamental structure of an exemplary sensor 10. A fluid channel 20 serves to guide and conduct a cell suspension across sensor elements 11 of a GMR (giant magnetoresistive) sensor. The cell suspension is supplied by microfluidic channel systems as known from U.S. Patent Publication No. 2011/0315635 A1. In the structure, the sensor elements form a first pair 12 and a second pair 13. In a manner known per se, both pairs 12, 13 are joined together in, in each case, a Wheatstone bridge in a parallel arrangement as depicted in FIG. 6. The first pair 12 generates a first sensor signal and the second pair 13 generates a second sensor signal. Both signals are generated when magnetically labeled cells or conglomerates in the fluid channel 20 move past the sensor elements 11, since the sensor elements 11 are capable of detecting magnetic fields in their immediate proximity. In an alternative embodiment, the sensor elements 11 may also be used directly for measurement without interconnecting them in a Wheatstone bridge. FIGS. 6 and 7 respectively depict the connection to form a Wheatstone bridge in a parallel arrangement, as used in the following examples, and in a diagonal arrangement. Here, the actual sensor elements 11 are interconnected electrically by conducting paths 61.

### Exemplary Embodiment #1

### Counting of Aggregates of Monocytes and/or Thrombocytes within a Whole Blood Sample

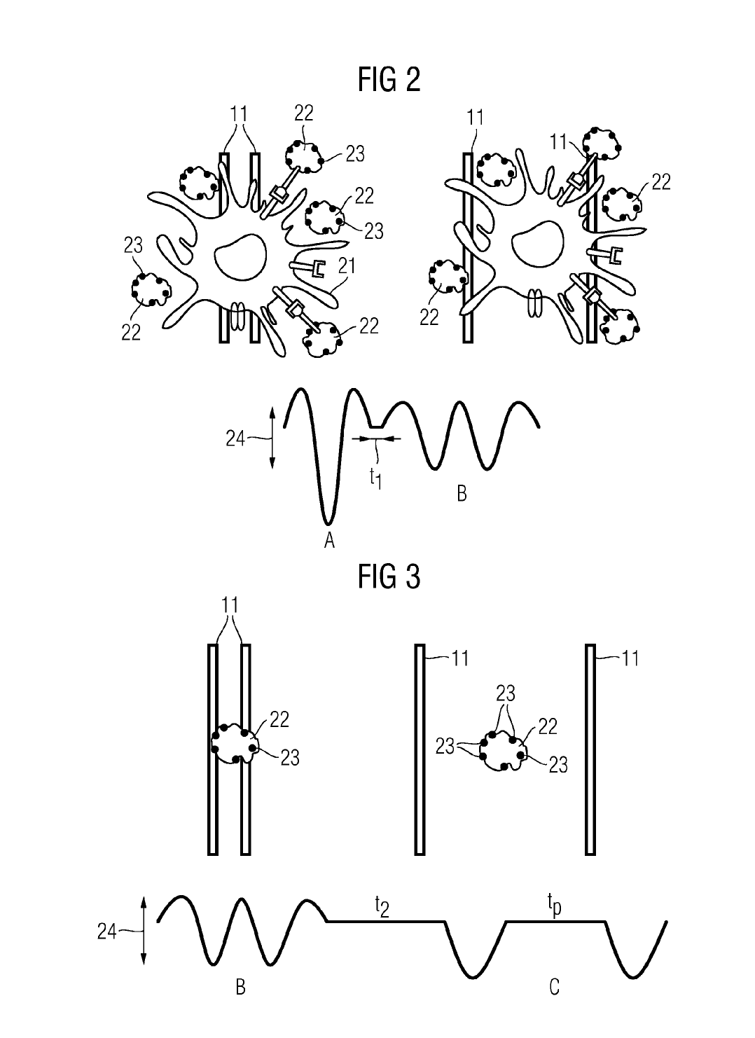

The first exemplary embodiment, which will be more particularly elucidated with the aid of FIGS. 2 to 5, addresses the specific counting of aggregates of monocytes 21 and/or thrombocytes 22 within a whole blood sample. In the embodiment, the thrombocytes 22 are labeled beforehand with superparamagnetic nanoparticles 23, which are in turn joined to a specific antibody. When the thrombocytes 22 interact with monocytes 21, they present antigens (for example, CD154) on their surface, which they would not present during the process of hemostasis. In this way, these thrombocytes 22 may, using specifically labeled nanoparticles 23, be distinguished from thrombocytes 22 not involved in blood coagulation. Thrombocytes 22 involved in blood coagulation are thus not labeled.

Because the thrombocytes 22 are labeled with superparamagnetic nanoparticles, the individual cells and aggregates are detectable by GMR sensor technology. If an individual thrombocyte 22, a monocyte/thrombocyte aggregate or a thrombocyte aggregate 41, 51 is conducted across the sensor, then characteristic signals are produced. If thrombocytes 22 react with monocytes 21 via specific antigen-antibody interactions, cell/cell aggregates having a mean size of about 25 μm are formed.

The sensor geometry of the sensor depicted in FIG. 1 is advantageously tailored to the measurement task. For instance, 2 μm is used as the spacing between the sensor elements 11 of the first pair 12, and additionally 25 μm as the spacing between the sensor elements 11 of the second pair 13, and 35 μm as the spacing between the closest sensor elements 11 of both pairs 12, 13.

FIG. 2 depicts an aggregate of a monocyte 21 and several thrombocytes 22 at two positions, over the first pair 12 and over the second pair 13. On the path across the two pairs 12, 13 of sensor elements 11 of the GMR sensor, the aggregate generates a signal sequence, as also depicted in FIG. 2. The characteristic signal A is generated upon coverage of the first pair 12. Signal A is substantially characterized by a brief deflection of high amplitude. The characteristic signal B is generated upon coverage of the second pair 13. Signal B is notable for a protracted signal profile having two similar peaks of a medium amplitude, which is used hereinafter as standard amplitude 24. The two peaks of signal B overlap as a result of the slight spacing between the sensor elements 11 of the first pair 12 and thus form the signal A. The larger spacing between the sensor elements 11 of the second pair 13 provides that these peaks do not overlap in this case. The described signals are separated in time by the time lag t1 owing to the flow velocity and thus the time required by the cell aggregate from the first pair 12 to the second pair 13.

Other types of cells and cell aggregates that may occur in this example may be clearly distinguished therefrom and from one another on the basis of their characteristic signals. FIG. 3 depicts the signal sequence that arises upon coverage of the sensor elements 11 by an individual labeled thrombocyte cell 22. Thus, coverage of the first pair 12 gives rise again to the characteristic signal sequence B, since the ratio between the sizes of cell and of the first pair 12 approximately matches the ratio between the sizes of aggregate of a monocyte 21 and several thrombocytes 22 and of the second pair 13. Upon coverage of the second pair 13, the individual labeled thrombocyte cell 22 generates a characteristic signal C in the form of two clearly separate deflections. The time lag t2 between the two signals is, in this case, clearly greater than the time lag t1. Therefore, a clear distinction between an individual thrombocyte cell 22 and an aggregate of such cells and a monocyte 21 is possible on the basis of the signals.

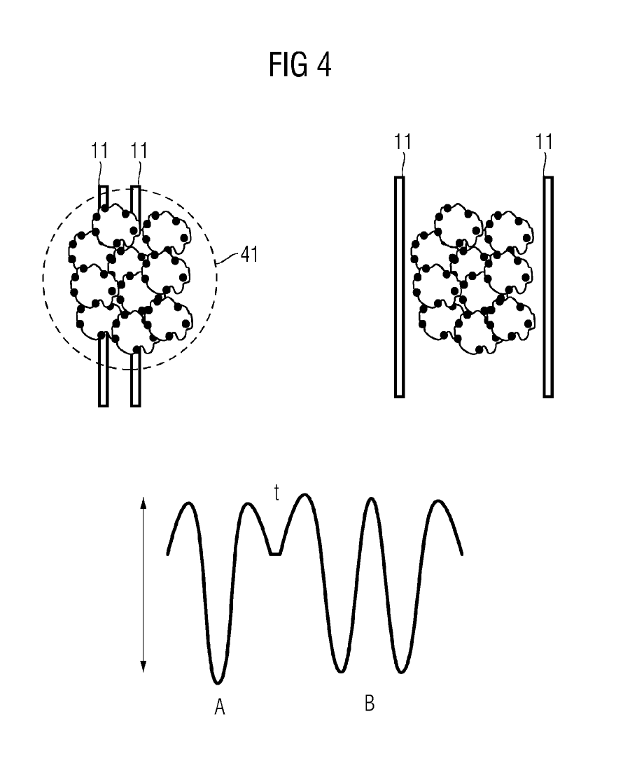

FIG. 4 depicts the signal sequence that arises upon coverage of the sensor elements 11 by a medium-sized conglomerate 41 of several labeled thrombocyte cells 22, (eleven cells in this example). Thus, coverage of the first pair 12 gives rise again this time to the characteristic signal sequence A having a peak of large amplitude, since the sensor elements 11 of the first pair 12, owing to their slight spacing, may not resolve the individual portions of the conglomerate 41. At a time lag of the size of about t1, a signal of the type of the characteristic signal B is produced, but this time with a substantially increased amplitude. Therefore, this conglomerate 41, without a monocyte cell 21, is also distinguishable from the aggregate with monocyte 21 on the basis of the amplitude of the signal of the second pair 13. Even clearer is the difference in relation to the signal sequence of an individual thrombocyte 22.

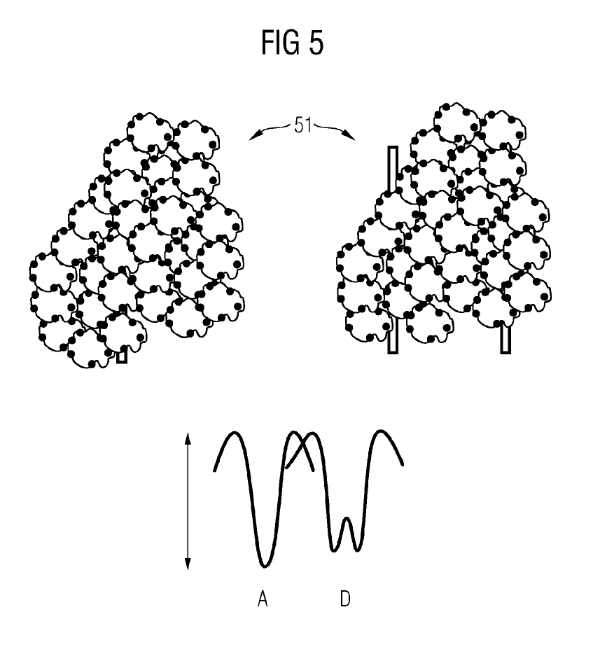

FIG. 5 depicts the signal sequence that arises upon coverage of the sensor elements 11 by a large conglomerate 51 of larger labeled thrombocyte cells 22, (over thirty cells in this example). Thus, coverage of the first pair 12 gives rise again this time to the characteristic signal sequence A having a peak of large amplitude, since the sensor elements 11 of the first pair 12, owing to their slight spacing, may not resolve the individual portions of the large conglomerate 51. Since the large conglomerate 51 is greater than the spacing between pairs 12, 13, there is no longer a time lag between the first and the second signal; instead, the signals overlap in parts. In the case of the second pair 13, a characteristic signal D of high amplitude arises owing to the fact that the large conglomerate 51 is greater than the spacing between the sensor elements 11 of the second pair 13. The signal sequence that comes about for the large conglomerate 51 is also distinguishable from the other types of cells and aggregates.

Therefore, the various cells and aggregates that occur may be distinguished on the basis of the following table. Here, it may be seen that, despite the labeling of only one cell type, different sizes and cell/cell aggregates may be measured by analysis of the different signal forms.

| | t1 | >t1 | <t1 |
|---|---|---|---|
| Standard amplitude 24 (second pair 13) | M/T | T | |
| Greater than standard amplitude 24 (second pair 13) | TT | | TTT |

first pair 12

| Second pair 13 | Signal A | Signal B |
|---|---|---|
| Signal B | M/T or TT | |
| Signal C | | T |
| Signal D | TTT | |

wherein:
M/T refers to an aggregate of monocyte 21 and thrombocytes 22
T refers to an individual thrombocyte cell 22
TT refers to a medium-sized aggregate 41 of thrombocytes 22
TTT refers to a large conglomerate 51 of thrombocytes 22.

Advantageously, what is thus done here is, firstly, the adaptation of the sensor geometry to the expected geometry or size of the analyte to be measured and, secondly, the setting of the spacing between two sensor strips, in order to distinguish immune cell/thrombocyte aggregates (diameter: 15-25 μm) from individual thrombocytes (2-5 μm) in the same sample. The spacing between the pairs 12,13 makes it possible to additionally rule out cell aggregates that are greater than the target structure, e.g., greater than about 25 μm in the present example. Furthermore, the resulting signal combinations make it possible to identify the cell or cell combination just measured.

The following acts are thus advantageously carried out or the advantages include (a) adaptation of the sensor geometry to the size of the analyte (e.g., magnetic particles such as metallic particles or magnetically labeled biochemical particles such as proteins or liposomes and also magnetically labeled biological particles such as animal cells, microorganisms and viruses). A time-of-flight measurement provides information about the size of the analyte. The advantages also include

(b) The arrangement of two sensors having different geometries allows the differentiation of particles of differing size and their composition by an exclusion method. In the method, the form of the individual signal and the temporal sequence of two signals is a specific criterion.

(c) The amplitude of the signal allows the differentiation of particle agglomerates of differing composition according to their magnetization. In this case, one component of the agglomerate is magnetically labeled (thrombocyte 22), whereas the other component remains unlabeled (monocyte 21). The unlabeled component influences the magnetization and size of the entire agglomerate.

(d) The measurement of an analyte may be carried out in complex liquids (including blood, urine, or secretions) without cleanup or dilution acts. An optical transparency is not required.

In the present first example, the cells used (e.g., primary phagocytes of the immune system) are between 15 and 30 μm in size. By contrast, the platelets are between 2 and 5 μm in size. This gives rise to a range for the spacings. For example, it is possible to use between 1 and 4 μm as the spacing between the sensor elements 11 of the first pair 12, and additionally between 20 and 30 μm as the spacing between the sensor elements 11 of the second pair 13 and between 30 and 40 μm as the spacing between the closest sensor elements 11 of both pairs 12, 13. The optimal geometry may be concretized experimentally.

### Exemplary Embodiment #2

### Labeling of Thrombocytes 22 within Cell Aggregates Together with Microorganisms (Bacteria, Viruses or Fungi/Yeasts)

The thrombocytes 22 gain increasing importance during the process of primary immune defense, where they interact in a supporting manner with immune cells or, in the event of ITP, also directly with foreign organisms such as bacteria, viruses or fungi and yeasts. A differentiation between these two causes of a case of thrombocytopenia (e.g., ITP or infection) may be crucial for a subsequent selection of a medicinal treatment.

In the event of a viral disease, thrombocytes 22 are also capable of ingesting and neutralizing them via phagocytosis. During this process, thrombocytes 22 are also capable of presenting MHC-I antigens (found especially on immune cells, but also on thrombocytes 22) on their surface to alert the immune system. A labeling of MHC-1 in blood and the counting of the cells may hint at a case of immunothrombocytopenia. In this case, large cells may be identified as immune cells and small cells as thrombocytes 22.

### Exemplary Embodiment #3

### Labeling of Endogenous Phagocytes of the Immune System within Aggregates with Large Cells (Circulating Tumor Cells, Inherent Immune Cells)

Endogenous phagocytes are capable of defanging circulating tumor cells identified as foreign bodies by the immune system, by phagocytosis (e.g., swallowing) and subsequent digestion. During this process, the diameter of a phagocyte becomes significantly greater on the one hand, and on the other hand, these cells also present specific antigens (e.g., MHC-1) on their surface during and after completion of the process. The magnetic labeling of these antigens, the determination of cell size and the subsequent counting of these cells provides indirect information about whether the number of circulating tumor cells is normal or increased.

### Exemplary Embodiment #4

### Measurement of Fibrin Formation on the Basis of Increasing Viscosity During Blood Coagulation

During hemostasis, the viscosity of blood increases owing to the formation of fibrin from fibrinogen. If blood is conducted through a microfluidic channel, the particles move free of friction with the fluid stream within the channel.

If fibrin is formed (the last step during blood coagulation), the viscosity of blood increases continuously until stoppage eventually occurs. If the viscosity increases and the flow velocity of the blood is slowed down, the velocity of particles within the blood also becomes increasingly lower. The slowing down of particles in coagulating blood may be used as a measure of its increasing viscosity and directly correlated with the increasing proportion of insoluble fibrin. Consequently, a time-of-flight measurement may also make it possible to measure the change in viscosity of the blood within the channel.

In this case, the time-of-flight measurement uses, for example, the spacing between the two pairs 12, 13 and the signals generated by the pairs when an analyte passes by.

### Exemplary Embodiment #5

### Magnetic Beads May be Used as Internal Standard for the Flow Velocity

Since the flow velocity of blood from different donors may vary owing to different initial viscosities, an internal standard allowing determination of the flow velocity at the start of each measurement may be introduced into the sample. Such a standard may include magnetic particles, which may differ from the analyte (very much smaller or very much larger) so that a mix-up with the analyte, (e.g., the actual cells or cell conglomerates), may be ruled out.

For exemplary embodiments 4 and 5, the initial pump output is the same.

In the exemplary embodiments, the starting point was a parallel arrangement of the sensor elements 11 in a Wheatstone bridge. In the arrangement, the individual sensor elements 11 of one pair 12, 13 provide temporally inverted signals, which, in the case of an overlap, leads to the signal sequences explained at the beginning depending on the analytes.

When using sensor elements 11 not interconnected to form a Wheatstone bridge or when using a diagonal arrangement of the sensor elements 11 in the Wheatstone bridge according to FIG. 7, the sensor signals of the sensor elements 11 are no longer temporally inverted, but instead follow one another without inversion. A temporal overlap of the signals likewise gives rise to characteristic signal forms according to the size of the particular analyte compared to the spacing between the sensor elements 11.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. An arrangement for quantifying cells while distinguishing between at least two different sizes of cell types, cell conglomerate types, or cell types and cell conglomerate types in a cell suspension, the arrangement comprising:
a magnetic field-sensitive sensor comprising a first pair of sensor elements and a second pair of sensor elements; and
a channel for guiding the cell suspension past the sensor elements,
wherein the sensor elements of the first pair comprise a first spacing of between half and double a first mean size of a first type of cell or cell conglomerate to be measured,
wherein the sensor elements of the second pair comprise a second spacing of between half and double a second mean size of a second type of cell or cell conglomerate to be measured, and
wherein a third spacing between a sensor element of the first pair and a sensor element of the second pair that are closest to one another is greater than the larger of the first and the second mean sizes.

2. The arrangement as claimed in claim 1, wherein the first spacing is between 1 and 4 μm, the second spacing is between 20 and 30 μm, and the third spacing is at least 30 μm.

3. The arrangement as claimed in claim 1, further comprising:
an evaluation unit for evaluating a first signal of the first pair and a second signal of the second pair, wherein the evaluation unit is configured to evaluate a time lag between the first and the second signal and an amplitude of the two signals.

4. The arrangement as claimed in claim 3, wherein the arrangement is configured for recording and distinguishing thrombocytes with magnetic labeling, immune cells associated with the thrombocytes, and conglomerates of the thrombocytes, wherein, for the coverage of one of the sensor pairs, a first signal amplitude and a first time lag between the signals is stored, and
(a) a conglomerate of the thrombocytes is recorded if the amplitude of the first and the second signal is greater than the first signal amplitude,
(b) an immune cell associated with the thrombocytes is recorded if the amplitude of the first signal is greater than the first signal amplitude first and second signal have a dissimilar amplitude, and
(c) an individual thrombocyte is recorded if the time lag is greater than the first time lag.

5. The arrangement as claimed in claim 1, wherein the sensor elements of the first pair and the second pair are in each case connected to form Wheatstone bridges.

6. The arrangement as claimed in claim 2, further comprising:
an evaluation unit for evaluating a first signal of the first pair and a second signal of the second pair, wherein the evaluation unit is configured to evaluate a time lag between the first and the second signal and an amplitude of the two signals.

7. The arrangement as claimed in claim 6, wherein the sensor elements of the first pair and the second pair are in each case connected to form Wheatstone bridges.

8. The arrangement as claimed in claim 6, wherein the arrangement is configured for recording and distinguishing thrombocytes with magnetic labeling, immune cells associated with the thrombocytes, and conglomerates of the thrombocytes, wherein, for the coverage of one of the sensor pairs, a first signal amplitude and a first time lag between the signals is stored, and
(a) a conglomerate of the thrombocytes is recorded if the amplitude of the first and the second signal is greater than the first signal amplitude,
(b) an immune cell associated with the thrombocytes is recorded if the amplitude of the first signal is greater than the first signal amplitude first and second signal have a dissimilar amplitude, and
(c) an individual thrombocyte is recorded if the time lag is greater than the first time lag.

9. The arrangement as claimed in claim 8, wherein the sensor elements of the first pair and the second pair are in each case connected to form Wheatstone bridges.

10. The arrangement as claimed in claim 2, wherein the sensor elements of the first pair and the second pair are in each case connected to form Wheatstone bridges.

11. The arrangement as claimed in claim 3, wherein the sensor elements of the first pair and the second pair are in each case connected to form Wheatstone bridges.

12. The arrangement as claimed in claim 4, wherein the sensor elements of the first pair and the second pair are in each case connected to form Wheatstone bridges.
